(19) United States
(12) Patent Application Publication
(10) Pub. No.: US 2026/0120791 A1
(43) Pub. Date: Apr. 30, 2026

(54) ENHANCED SYSTEM FOR TREATING A BIOTECHNOLOGICAL FLUID USING AN ADAPTIVE SOFTWARE

(71) Applicant: MERCK PATENT GMBH, Darmstadt (DE)

(72) Inventors: Philippe EBERHARD, Molsheim (FR); Rene REINBIGLER, Molsheim (FR); Denis DUBRET, Molsheim (FR); Steven MILLER, Molsheim (FR)

(73) Assignee: MERCK PATENT GMBH, Darmstadt (DE)

(21) Appl. No.: 18/690,804

(22) PCT Filed: Sep. 19, 2022
(86) PCT No.: PCT/EP2022/075874

§ 371 Date: Mar. 11, 2024

(30) Foreign Application Priority Data
Sep. 22, 2021 (EP) 21306311.8

(51) Int. Cl. G16B 15/00; G16B 40/20

(52) U.S. Cl. CPC G16B 15/00; G16B 40/20

(57) ABSTRACT

The present application relates to biotechnological fluid treating, such as biopharmaceutical liquids in order to obtain products such as monoclonal antibodies, vaccines or recombinant proteins, and particularly to a system for treating a biotechnical fluid, having a bioprocess machine and at least one bioprocess machine helper, configured to connect to each other and each comprising a controller, in turn comprising a machine-to-machine communication tool configured for connecting to a network, wherein parts of the bioprocess machine helper are simulated and used to enhance the behaviour of the disclosed system.

### FIELD OF THE INVENTION

The invention relates to biotechnological fluid treating, such as biopharmaceutical liquids in order to obtain products such as monoclonal antibodies, vaccines or recombinant proteins.

### BACKGROUND ART

It is known that biotechnological fluids such as biopharmaceutical liquids are in general obtained first by a treatment such as cell or micro-organism culture in a bioreactor and that they must then be further treated to achieve the required characteristics of homogeneity, purity, concentration, absence of viruses, etc.

These treatments are conventionally carried out in dedicated installations, with stainless steel pipes and other components such as tanks and filter housings, which necessitate operations before and after the actual treatment, which are relatively onerous, in particular, operations for cleaning after use.

Within the last few years, these treatments have alternatively been carried out in installations in which the components in contact with the liquid are single-use components, for avoiding cleaning operations.

For instance, European patent applications EP 2130903 A1 and EP2208534 A1 disclose installations including disposable elements, for the most part flexible (“Flexware™ products”), including the treated liquid collecting bag and the circuit sections, even the filter element, and permanent or reusable elements (“hardware”) accommodated in two or more carts, so that an installation for treating a biotechnological fluid can be assembled simply by equipping the carts with the disposable elements, whereas the post-treatment steps are essentially the removal and discarding of the disposable elements.

Other known installations are using the same approach with the reusable elements or certain reusable elements which are not accommodated in carts.

In general, the main reusable element of an installation for treating a biotechnological fluid is a bioprocess machine having a biotechnological fluid treater configured for modifying at least one physico-chemical or biological property of the biotechnological fluid, for example its pH, DO (Dissolved Oxygen), homogeneity, purity, concentration, presence or absence of predetermined micro-organisms, e.g., viruses and/or other pathogens.

Further to the biotechnological fluid treater, the bioprocess machine has a digital controller for controlling the biotechnological fluid treater and most often the digital controller is able to pilot the fluid treater so that the machine can carry out automatically a customized version of the treatment, generally called a recipe.

### SUMMARY OF THE INVENTION

The invention is directed to further ease the setting up of installations for treating a biotechnological fluid.

The invention provides accordingly a system for treating a biotechnological fluid, having the following system devices of a bioprocess machine having: a physical biotechnological fluid treater which is configured for modifying at least one physico-chemical or biological property of said biotechnological fluid and a digital controller for controlling said physical biotechnological fluid treaters; and at least one physical bioprocess machine helper having: a physical biotechnological fluid treater helper configured for being coupled to said biotechnological fluid treaters and a digital controller for controlling said biotechnological fluid treater helpers; and/or at least one software-based bioprocess machine helper created by an adaptive device creator a software-based bioprocess machine match helper for selecting a suitable software-based bioprocess machine helper wherein said digital controller of said bioprocess machine and said digital controller of said machine helper each include at least one capability manager, a machine to machine communication tool (MtoM communication tool), and a discovery negotiation pairing manager (DNP manager); each said MtoM communication tool is configured for connecting to a network; the DNP manager of said bioprocess machine and the DNP manager of said machine helpers are configured for cooperating over said network for establishing a paired condition; wherein the capability manager of the bioprocess machine and the capability manager of the machine helpers are configured such that in said paired condition they organize the provision and/or consummation of the respective capabilities by either said bioprocess machine or said machine helpers; said software-based bioprocess machine helper is configured by said suitable adaptive device creator to predict or compute the behaviour of said biotechnological fluid using physico-chemical or biological properties and/or external data, analyse the result and extend the capabilities of said physical bioprocess machine.

The physical coupling between the machine helper (through the treater helper) and the bioprocess machine (through the fluid treater) is at least for enabling the treater helper to assist the fluid treater in modifying the at least one physico-chemical or biological property of the biotechnological fluid, for example by pumping or exerting another physical action on the fluid or by sensing a physico-chemical or biological quantity of the fluid, such as pH or Dissolved Oxygen (DO).

In the system according to the invention, the machine helper has a digital controller with a machine to machine communication tool so as to communicate with the bioprocess machine through a network enabling machine to machine communications, despite the physical coupling between the machine helper and the bioprocess machine which would have enabled relatively easily a dedicated communication channel such as a wired serial or parallel link, which could be operational merely by plugging connectors when carrying out the physical coupling.

The invention is based on the observation that despite the need to carry out pairing through the network further to the physical coupling, such pairing through the network can in fact ease the setting up of installations for treating a biotechnological fluid because such pairing can be used for automatically reconfiguring accordingly the bioprocess machine (with the provided capability) and the machine helper (with the consumed capability).

With such automated reconfiguring, adding a machine helper is very convenient and can be done even during a batch process so that the system according to the invention further offers excellent flexibility.

Another important part of the invention is that at least one software-based bioprocess machine helper component can be added to the system. It is established by selecting one or more of those software-based helper components via the match helper and then created by a specific software called the adaptive device creator. This software-based bioprocess machine helper then uses data about the biotechnological fluid to predict and/or compute its behaviour. With this simulated behaviour it is possible to test and enhance the functionality of physical bioprocess machine helper and therefore the performance of the whole bioprocess machine. The software-based bioprocess machine helper can be used in an existing bioprocess machine with real bioprocess machine helpers or it can be the only bioprocess machine helper there, whatever suits the use case best. It is also possible to use more than one software-based bioprocess machine helper in the system. Their configuration depends on the respective use case as well.

According to possible advantageous features the invented system comprises that:
the adaptative device creator and/or at least one software-based bioprocess machine helper is hosted either by a remote computer which transfers said created software-based bioprocess machine helper to its respective digital controller or is hosted directly as a local instance on said respective digital controller;
said at least one software-based bioprocess machine helper is configured to have the same behaviour than its physical counterpart;
said digital controller of said bioprocess machine includes a file containing a description of each said interface function which can be a said provided capability and said digital controller of said machine helper includes a file containing a description of each said interface function which can be a said consumed capability;
the DNP manager of said bioprocess machine and the DNP manager of said machine helper are configured for cooperating over said network for establishing a paired condition;
said system devices include a plurality of said machine helpers and the DNP manager of said bioprocess machine is configured for establishing a said paired condition simultaneously with at least one said machine helper;
said system devices include a first said bioprocess machine, a second said bioprocess machine and a plurality of said machine helpers; and the DNP manager of at least one said machine helper is configured for establishing a said paired condition either with said first bioprocess machine or with said second bioprocess machine;
is having a first said machine helper in which the consumed capability is an interface function controlling and/or displaying an operating parameter of the treater helper of said first machine helper; and having a second said machine helper in which the consumed capability is an interface function displaying a physico-chemical or biological quantity of the fluid sensed by the treater helper of second machine helper, wherein the first machine helper is a pump in which the consumed capability is an interface function controlling and/or displaying a speed of the pump and the second machine helper is a pH sensor or a flow sensor in which the consumed capability is an interface function displaying a pH of said fluid or a flow of said fluid;
each said MtoM communication tool is configured for connecting to a said network which is a network with Internet Protocol, such as Ethernet, Wi-Fi, Bluetooth or cellular 5G.

The described system can vary in different embodiments depending on which function the system should perform. One of those embodiments of the inventions comprises of a method for operating a system for treating a biotechnological fluid according to any one of the preceding claims, the following steps of:
Identifying relevant process context and required capabilities of said physical biotechnological fluid treater and use it to select at least one suitable software-based bioprocess machine helper via said software-based bioprocess machine match helper; creating the selected at least one software-based bioprocess machine helper via said adaptative device creator; adding all software-based devices to the system and pairing the at least one software-based bioprocess machine helper with the respective bioprocess machine via the DNP manager; predicting and/or computing the behavior of said biotechnological fluid in said physical biotechnological fluid treater using its at least one physico-chemical or biological properties and/or external data via said software-based bioprocess machine helper; and analyzing the data and with the results adapting the configuration of said physical biotechnological fluid treater and extending the capabilities of said physical bioprocess machine helper via said software-based bioprocess machine helper, comprising.

According to advantageous features the invented method carries out several additional steps wherein:
said software-based bioprocess machine helper uses artificial intelligence algorithms, like machine learning models, neural networks and others, and/or non-self-learning tools, like calculation models, for its predictions, computations and data analyses.
the artificial intelligence algorithms are trained with either external data or the collected data about said at least one physico-chemical or biological properties from said biotechnological fluid in said physical biotechnological fluid treater.
the software-based bioprocess machine helper uses Smart Data for its predictions, computations and data analyses.
the adaptive device creator uses a contextualization mechanism on instances of said Smart data.

### BRIEF DESCRIPTION OF THE DRAWINGS

The description of the invention continues now with a detailed description of example embodiments given hereinafter by way of non-limiting illustration and with reference to the appended drawings. In the drawings:

FIG. 1 schematically illustrates a production area in a bioprocess production plant or laboratory, in which is located a bioprocess machine forming part of a system for treating a biological fluid;

FIG. 2 schematically illustrates a storage area of the bioprocess production plant or laboratory, in which are located a plurality of bioprocess machine helpers of the system for treating a biological fluid, the bioprocess machine helpers being configured for being associated to the bioprocess machine illustrated on FIG. 1;

FIG. 3 is a diagram illustrating the bioprocess machine and one of the bioprocess machine helpers, together with a network through which the bioprocess machine and the bioprocess machine helper cooperate for establishing a paired condition;

FIG. 4 shows the graphical user interface of a bioprocess machine which is a mixer, in stand-alone condition;

FIG. 5 shows at the top the graphical user interface of the mixer when paired with a bioprocess machine helper which is a pH sensor, and shows at the bottom the graphical user interface of the pH sensor, respectively on the left in stand-alone condition and on the right when paired with the mixer;
FIG. 6 schematically illustrates at the top a bioprocess machine helper which is a pump together with a bioprocess machine helper which is a flow sensor, the pump and the flow sensor being physically coupled; and also shows at the middle the graphical user interface of the pump, respectively on the left in stand-alone condition and on the right when paired with the flow sensor; and also shows at the bottom the graphical user interface of the flow sensor, respectively on the left in stand-alone condition and on the right when paired with the pump;
FIG. 7 is an exemplary schematic representation of the GUIs adapting when the process machine and a machine helper are being paired, showing at the top the graphical user interface of the mixer, respectively on the left when paired with the pH sensor and on the right when paired with the pH sensor and paired with the pump paired with the flow sensor; and also showing at the middle the graphical user interface of the pump, respectively on the left when paired with the flow sensor and on the right when paired with the flow sensor and paired with the mixer; and also showing at the bottom the graphical user interface of the flow sensor, respectively on the left when paired with the pump, and on the right when paired with the pump paired with the mixer;

FIG. 8 is an exemplary schematic representation of the GUIs adapting when the process machine and a machine helper are being unpaired, showing at the top the graphical user interface of the mixer, respectively on the left when paired with the pH sensor and paired with the pump paired with the flow sensor, and on the right when paired with the pH sensor; and also showing at the middle the graphical user interface of the pump, respectively on the left when paired with the flow sensor and paired with the mixer and on the right when paired with the flow sensor; and also showing at the bottom the graphical user interface of the flow sensor, respectively on the left when paired with the pump paired with the mixer and on the right when paired with the pump; and
FIG. 9 shows the three method steps which needs to be performed when using virtual, software based components in the invented system
FIG. 10 shows the whole invented method for operating a system for treating a biotechnological fluid including the usage of virtual, software based components

### DETAILED DESCRIPTION OF EXAMPLE EMBODIMENTS

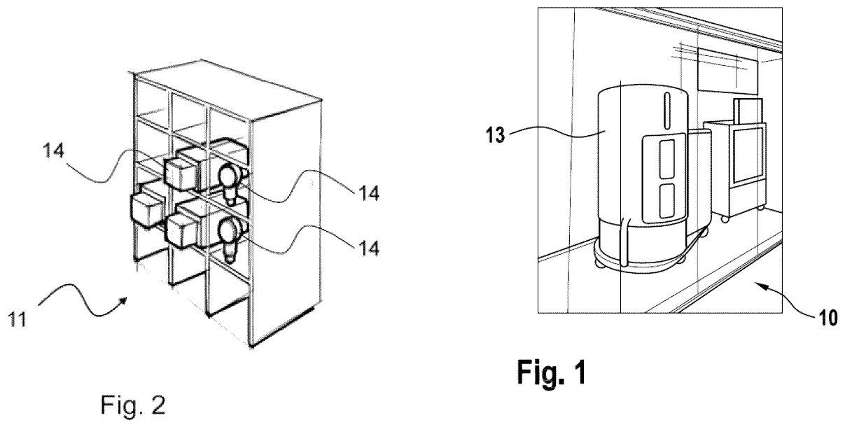

FIGS. 1 and 2 illustrate the production area 10 and the storage area 11 of a bioprocess production plant or laboratory in which are available system devices of a system for treating a biotechnological fluid according to the invention.

### Description of the System Devices: Bioprocess Machine and Bioprocess Machine Helper

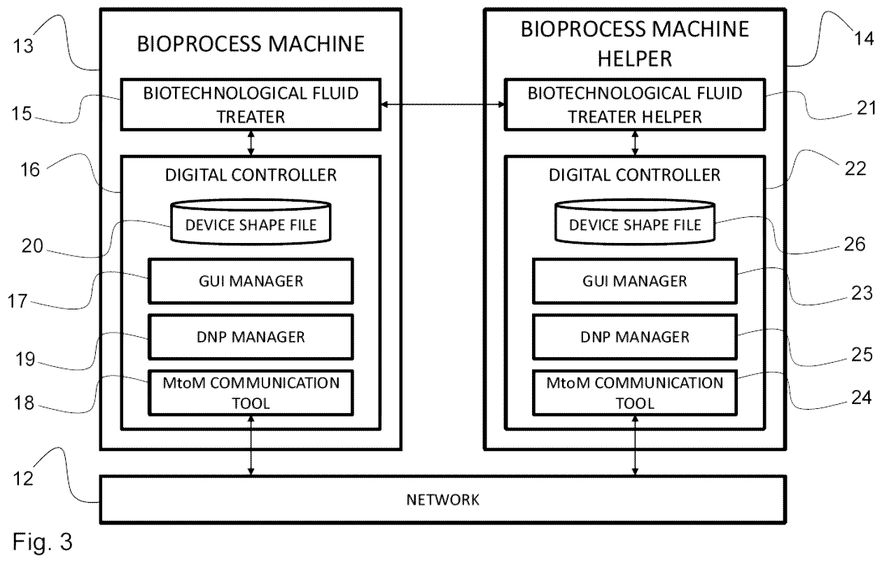

Each system device includes digital processing units (microprocessor and/or microcontroller, memory, network connectivity) and is configured to be wire or wireless connected to a network 12 (FIG. 3) supporting Internet Protocol (IP).

For instance, wire connection is through Ethernet and wireless connection is through Wi-Fi, Bluetooth or cellular such as 5G.

This system has a bioprocess machine 13 and a plurality of bioprocess machine helpers 14. The bioprocess machine 13 alone or associated with one or more machine helpers 14 can be set up for becoming an installation for treating a biotechnological fluid.

As shown on FIG. 3, the bioprocess machine 13 has a biotechnological fluid treater 15 and a digital controller 16.

The biotechnological fluid treater 15 is configured for modifying at least one physico-chemical or biological property of the biotechnological fluid, for example its pH, DO (Dissolved Oxygen), homogeneity, purity, concentration, presence or absence of predetermined micro-organisms such as viruses.

The digital controller 16 is configured for controlling the biological fluid treater 15, as shown by a bidirectional arrow on FIG. 3. Here, the digital controller 16 is able to pilot the fluid treater 15 so that the machine 13 can carry out automatically a customized version of the treatment, generally called a recipe.

The digital controller 16 includes at least one capability manager, like a graphical user interface (GUI) manager 17, a reporting manager or a recipe manager, for handling different tasks. In the preferred embodiment a graphical user interface (GUI) manager 17 is used. Furthermore the digital controller 16 includes a machine to machine (MtoM) communication tool 18, and a discovery negotiation pairing (DNP) manager 19. The digital controller 16 also includes a file 20 called Device Shape which contains a description of certain interface functions of a GUI which can be displayed by the GUI manager 17.

It should be noted that the term “file” is here to be taken in a broad sense, namely encompassing any structured data container, including a folder and/or a database.

The bioprocess machine helper 14 has a biotechnological fluid treater helper 21 and a digital controller 22.

The treater helper 21 is configured for being physically coupled to the fluid treater 15, as shown by a bidirectional arrow on FIG. 3. The physical coupling is for enabling the treater helper 21 to assist the fluid treater 15 in modifying at least one physico-chemical or biological property of the biotechnological fluid, for example by pumping or exerting another physical action on the fluid or by sensing a physico-chemical or biological quantity of the fluid, such as pH or DO.

For instance, if the bioprocess machine 13 is a mixer, the biotechnological fluid treater 15 includes the tank and the agitator; if the machine helper 14 is a pump, the biotechnological fluid treater helper 21 includes the fluid driving member(s) such as the roller(s) of a peristaltic pump; and if the machine helper 14 is pH or flow sensor, the biotechnological fluid treater helper 21 includes respectively a pH probe and a flow probe.

The physical coupling between the fluid driving member(s) (treater helper 21 of the pump) and the tank+agitator (fluid treater 15 of the mixer) involves pipes and also holders for maintaining in a predetermined relative position the fluid driving member(s) and the tank+agitator. For instance, such holders are carried out through mounting of the pump on the same or similar framework as the mixer or through a cart, on which is mounted the pump, such cart being maintained in a fixed position with respect to the mixer.

Similarly, the pH probe or flow probe must interact with the fluid and be maintained in place.

Generally speaking, the physical coupling involves an interaction with the fluid (with or without contact, see the rollers of a peristaltic pump which are not in contact with the fluid or an IR probe of a temperature sensor which is not in contact with the fluid); and holders for maintaining the treater helper 21 with respect to the fluid treater 15.

The digital controller 22 is configured for controlling the treater helper 21, as shown by a bidirectional arrow on FIG. 3.

The digital controller 22 has the same architecture as the digital controller 16: the digital controller 22 includes a GUI manager 23, a MtoM communication tool 24, and a DNP manager 25. The digital controller 22 also includes a file 26 called Device Shape which contains a description of certain interface functions of a GUI which can be displayed by the GUI manager 23.

In each system device 13 or 14, the GUI manager 17 or 23 allows to display a GUI such as a Process and Instrumentation Diagram (P&ID) locally on an interactive screen or remotely on a device having an interactive screen, for e.g., a tablet or smartphone.

In each system device 13 or 14, the MtoM communication tool 18 or 24 is configured for connecting to the network 12, as shown by bidirectional arrows on FIG. 3.

The DNP manager 19 of the bioprocess machine 13 and the DNP manager 25 of the machine helper 14 are configured for cooperating over the network 12 for establishing a paired condition.

Each system device 13 or 14 can be used as a stand-alone device or paired with an appropriate other system device. The GUI manager 17 or 23 of each system device 13 or 14 is configured for undergoing an adaptation of the graphical use interface (GUI) when the system device transitions from a stand-alone (not paired) condition to a paired condition and vice-versa.

For instance, if the machine helper 14 is a pump which can be paired with a bioprocess machine 13, in stand-alone condition of the pump its GUI enables the user to control the pump so that it is possible for the user to utilize the pump as a stand-alone unit for tasks such as transferring a liquid from one tank to another; whereas in paired condition of the pump with the bioprocess machine 13 the GUI of the pump no longer enables the user to control the pump, only the GUI of the bioprocess machine 13 enables to control the pump.

Still for instance, if the machine helper 14 is a sensor which can be paired with a bioprocess machine 13, such sensor sensing a physico-chemical or biological quantity of a biotechnological fluid, in stand-alone condition of the sensor its GUI displays the sensed quantity so that it is possible for the user to utilize the sensor as a stand-alone unit, whereas in paired condition of the sensor with the bioprocess machine 13 the GUI of the sensor displays only a message, such as “CONNECTED”, informing that the sensor is in paired condition and the GUI of the bioprocess machine 13 displays the quantity sensed by the sensor.

Generally speaking, the GUI manager 17 of the bioprocess machine 13 and the GUI manager 23 of the machine helper 14 are configured such that in paired condition:
the GUI manager 17 of the bioprocess machine 13 has at least one provided capability that it does not have when not in paired condition, said provided capability being an interface function controlling and/or displaying an operating parameter of the treater helper 21 or displaying a physico-chemical or biological quantity of the fluid sensed by the treater helper 21; and
the GUI manager 23 of the machine helper 14 has at least one consumed capability which is modified with respect to when not in paired condition, wherein: if said provided capability is said interface function controlling and/or displaying an operating parameter of the treater helper 21 said consumed capability is an interface function controlling and/or displaying said operating parameter, and if said provided capability is said interface function displaying a physico-chemical or biological quantity of said fluid sensed by the treater helper 21 said consumed capability is an interface function displaying said physico-chemical or biological quantity.

### Capabilities

The interface functions described in the Device Shape file 20 or 26 of the different system devices 13 and 14 are either of a first type or of a second type.

The interface functions of the first type are interface functions that the GUI manager 17 or 23 of the system device 13 or 14 does not have when the system device is not paired with an appropriate other system device but with which the GUI manager 17 or 23 is supplemented when the system device 17 or 23 is paired with the appropriate other system device.

In practice, interface functions of the first type are present but disabled when the system device 13 or 14 is not paired with an appropriate other system device and enabled when the system device 13 or 14 is paired with an appropriate other system device.

When enabled, each such interface function controls and/or displays an operating parameter of a paired system device or displays a quantity sensed by a paired system device, said quantity being a physico-chemical or biological quantity of the biotechnological fluid being treated.

For mere language convenience, such interface functions are designated herein as a “capability” and when enabled as a “provided capability.”

The interface functions of the second type are interface functions that the GUI manager 23 of a system device which is a machine helper 14 has in an original form when the system device is not paired with an appropriate other system device and in a modified form when the system device is paired with the appropriate other system device.

When in original form, each such interface function controls and/or displays an operating parameter of the system device or displays a quantity sensed by a paired system device, said quantity being a physico-chemical or biological quantity of the biotechnological fluid being treated. When in modified form, each such interface function is for instance as in original form but with an additional display of an indication that the system device is paired with an appropriate other system device, or the original form is replaced by an indication that the system device is paired with an appropriate other system device, such indication being for instance an icon, a message or the absence of display.

For mere language convenience, such interface functions are designated herein as a “capability” and when in modified form as a “consumed capability.”

It should be noted that the Device Shape file 20 of the bioprocess machine 13 contains a description of interface functions of its GUI manager 17 which are all of the first type; and that the Device Shape file 26 of a machine helper 14 contains a description of at least one interface function of its GUI manager 23 which is of the second type.

It should be further noted that in the Device Shape files 20 and 26, each capability description has a feature named “Role” identifying whether the capability is of the first type or of the second type.

For the first type, the Role feature is at “Consumer”, with reference to the corresponding capability in the paired system device which becomes a “consumed capability” in paired condition. A system device 13 or 14 having a capability with the Role feature at “Consumer” is mentioned herein as a being a capability consumer.

For the second type, the Role feature is at “Provider”, with reference to the corresponding capability in the paired system device which becomes a “provided capability” in paired condition. A system device 14 having a capability with the Role feature at “Provider” is mentioned herein as a being a capability provider.

It should be noted that there is always a correspondence between a provided capability and a consumed capability.

When the provided capability (in the capability consumer 13 or 14) is an interface function controlling and/or displaying an operating parameter of the capability provider, the consumed capability (in the capability provider 14) is an interface function controlling and/or displaying this operating parameter. For instance, if the provided capability in a mixer is a Start/Stop control of a paired pump, the consumed capability in the paired pump is a Start/Stop control of this pump.

When the provided capability (in the capability consumer 13 or 14) is an interface function displaying a physico-chemical or biological quantity of the biotechnological fluid sensed by the capability provider 14, the consumed capability (in the capability provider 14) is an interface function displaying this physico-chemical or biological quantity. For instance, if the provided capability in a mixer is a display of the pH of the biotechnological fluid sensed by a paired pH sensor, the consumed capability in the paired pH sensor is the display of the sensed pH.

Corresponding provided capability and consumed capability are referred to herein as “shared capabilities.”

For each system device 13 or 14, the Device Shape file 20 or 26 is deployed at design time and can be updated, at runtime, and during the life of the system device, allowing to extend the list of capabilities a capability consumer can consume or a capability provider can provide. This renders it possible to make the system devices contribute to a new platform feature, without modifying (and then requalifying) the software package installed in the system devices.

Now will be described examples of system devices 13 and 14 and how an installation for treating a biotechnological fluid is set up with these system devices.

The example of bioprocess machine 13 is a mixer (capability consumer). The examples of bioprocess machine helpers 14 are a pH sensor (capability provider), a flow sensor (capability provider) and a pump (together capability consumer and capability provider).

### Description of the Mixer

The mixer includes a tank, an agitator within the tank and two inlets allowing to connect pipes that can be used to fill the tank.

The tank, agitator and inlets form a biotechnological fluid treater 15.

To be operational, the mixer requires connection to at least one pump to flow the biotechnological fluid through one of the inlets.

The mixer includes digital processing units including an industrial Programmable Logic Controller (PLC) and an industrial PC.

The PLC is dedicated to the real time control and monitoring of the different equipment modules to which it is connected (for example, in a wireless manner or by wire), such as the agitator and valves opening or closing the inlets.

On the industrial PC is installed a software package and stored the file 20 called Device Shape.

The digital processing units, the installed software package and the stored Device Shape file 20 form a digital controller 16.

The installed software package includes: a DNP manager 19; a MtoM communication tool 18 having here an OPC UA server and an OPC UA client to support data exchanges with other system devices; and a GUI manager 17 that allows to display a process and instrumentation diagram (P&ID) locally on an interactive screen or remotely on a device having an interactive screen, for e.g., a tablet or smartphone.

The file 20 called Device Shape contains a description of four interface functions which are provided capabilities when enabled.

### Description of the Capabilities Which Can Be Utilized by the Software Package of the Mixer

As just mentioned, there are four such capabilities, respectively Interface Function 1, Interface Function 2, Interface Function 3 and Interface Function 4.

### Interface Function 1

When enabled, Interface Function 1 supplements the P&ID GUI with process data provided by paired system devices, whatever the paired system devices are.

This capability description means: the mixer, as a capability consumer with graphic skills, is able to display process value from several paired system devices if these paired system devices provide at least the process value, the process value name and unit and optionally the number of significant decimal digits using the OPC UA standard. No restriction or condition are imposed for the negotiation or pairing.

In such a variant, the capability description further means that the mixer is able to use the process value range if provided by a paired system device, to propose supplementary types of display (e.g. gauge, . . . ).

### Interface Function 2

When enabled, Interface Function 2 adapts the P&ID GUI to show if the mandatory expected pump has been paired and supplements the P&ID GUI with control icons and displays of operating parameters of the paired pump.

This capability description means: the mixer, as a capability consumer with control skills, requires a system device which is a pump to be mandatorily paired to achieve the role defined for the pump connected to the inlet 1. To be paired, the system device will mandatorily have to make available a Start/Stop command and provide its current started status. The mixer will be able to control and monitor the pump speed if provided by the paired system device. The confirmation by the operator is required during the pairing procedure. Once paired, the mixer will have the exclusive usage of the system device which is a pump.

In such a variant, the capability description further means that the mixer will be able to display the minimum and maximum values of a speed range if provided by the paired pump, in order to guide the operator when setting the pump speed.

### Interface Function 3

When enabled, Interface Function 3 adapts the P&ID GUI to show if the planed optional pump has been paired and supplements the P&ID GUI with control icons and displays of operating parameters of the paired pump.

This capability description means: the mixer, as a capability consumer with control skills, is able to control an optional system device which is a pump to achieve the predefined role for the pump connected to the inlet 2. To be paired, the system device will mandatorily have to make available a Start/Stop command and provide its current started status. The mixer will be able to control and monitor the pump speed if provided by the paired system device. The confirmation by the operator is required during the pairing procedure. Once paired, the mixer will have the exclusive usage of the system device which is a pump.

In such a variant, the capability description further means that the mixer will be able to display the minimum and maximum values of a speed range if provided by the paired pump, in order to guide the operator when setting the pump speed.

### Interface Function 4

When enabled, Interface Function 4 adapts the P&ID GUI to show any other optional paired pump and supplements the P&ID GUI with control icons and displays of operating parameters of the paired pump.

This capability description means: the mixer, as a capability consumer with control skills, is able to control any other optional system device that is a pump. No mandatory properties are expected. The mixer will be able to start/stop the pump and to control and monitor the pump speed if provided by the paired system device. No confirmation by the operator is required during the pairing procedure. Once paired, the mixer will have the exclusive usage of the system device which is a pump.

It should be noted that not all interface functions of the GUI manager 17 of the mixer have been described here. The interface functions regarding the equipment modules in the mixer (agitator, controls of inlet valves, . . . ) are not described here. Only interface functions disabled when the mixer is not paired with the appropriate system device and enabled when the mixer is paired with the appropriate system device are described and at least other such interface function may be included in the GUI manager 17 of the mixer.

It should be further noted that the capabilities Interface Function 2, Interface Function 3 and Interface Function 4 illustrate three levels of capabilities that can become provided capabilities, namely predefined and mandatory capability such as Interface Function 2, predefined and optional capability such as Interface Function 3 and optional supplementary capability such as Interface Function 4.

### Description of the pH Sensor

The pH sensor includes a probe for sensing the pH of the biotechnological fluid. The probe forms a biotechnological fluid treater helper 21.

The pH sensor includes digital processing units including a microprocessor and/or a microcontroller, a memory and network connectivity.

The processing units are configured for controlling and monitoring the probe for sensing the flow, to which they are electrically wired.

On the processing units is installed a software package and stored a file 26 called Device Shape.

The processing units, the installed software package and the stored Device Shape file 26 form a digital controller 22.

The installed software package has the same architecture as the software package installed in the mixer, the software package installed in the pH sensor including: a DNP manager 25; a MtoM communication tool 24 having here an OPC UA server and an OPC UA client to support data exchanges with other system devices; and a GUI manager 23 that allows to display a GUI remotely on a device having an interactive screen, for e.g., a tablet or smartphone.

The file 26 called Device Shape contains a description of one interface function which is a consumed capability when in modified form.

### Description of the Capability Which Can Be Utilized by the Software Package of the pH Sensor

When in modified form the single interface function described in the Device Shape file of the pH sensor keeps the display of the current value measured by pH sensor and a trend curve representing the variation of the pH in time and adds an indication that the pH sensor is paired with an appropriate other system device.

This capability description means: the pH sensor, as a capability provider, is able to provide a pH (and only a pH) process value display dataset to paired system devices using the OPC UA standard. The dataset includes a process value, its name and its unit. The OPC UA tag values for each data item is specified allowing the paired system devices to read these values with an OPC UA client. No specific restriction or condition is imposed for the negotiation or the pairing.

In a variant, the dataset further includes at least one of the minimum value or the maximum value of a range of values within which the pH process value is expected to be found.

### Description of the Flow Sensor

The flow sensor includes a probe for sensing the flow of biotechnological fluid. The probe forms a biotechnological fluid treater helper 21.

The flow sensor includes digital processing units including a microprocessor and/or a microcontroller, a memory and network connectivity.

The processing units are configured for controlling and monitoring the probe for sensing the flow, to which they are electrically wired.

On the processing units is installed a software package and stored a file 26 called Device Shape.

The processing units, the installed software package and the stored Device Shape file 26 form a digital controller 22.

The installed software package has the same architecture as the software package installed in the mixer, the software package installed in the flow sensor including: a DNP manager 25; a MtoM communication tool 24 having here an OPC UA server and an OPC UA client to support data exchanges with other system devices; and a GUI manager 23 that allows to display a GUI remotely on a device having an interactive screen, for e.g., a tablet or smartphone.

The file 26 called Device Shape contains a description of one interface function which is a consumed capability when in modified form.

### Description of the Capability Which Can Be Utilized by the Software Package of the Flow Sensor

When in modified form the single interface function described in the Device Shape of the flow sensor replaces the display of the current value measured by the flow sensor and a trend curve representing the variation of the flow in time by the display of an indication that the flow sensor is paired with an appropriate other system device.

This capability description means: the flow sensor, as a capability provider, is able to provide a flow (and only a flow) process value display dataset to paired system devices using the OPC UA standard. The dataset includes a process value, its name and its unit. The OPC UA tag values for each of the data is specified allowing the paired system devices to read these values with an OPC UA client. No specific restriction or condition is imposed for the negotiation or the pairing.

In a variant, the dataset further includes at least one of the minimum value or the maximum value of a range of values within which the flow process value is expected to be found.

### Description of the Pump

The pump includes members for acting on the fluid for driving it, for instance the rollers of a peristaltic pump, and a motor for driving such members. The driving motor and the driven members acting on the fluid form a biotechnological fluid treater helper 21.

The pump includes digital processing units including a microprocessor and/or a microcontroller, a memory, and network connectivity.

The processing units are configured for controlling and monitoring the motor, to which they are electrically wired.

On the processing units is installed a software package and stored a file 26 called Device Shape.

The processing units, the installed software package and the stored Device Shape file 26 form a digital controller 22.

The installed software package has the same architecture as the software package installed in the mixer: the software package installed in the pump includes: a DNP manager 25; a MtoM communication tool 24 having here an OPC UA server and an OPC UA client to support data exchanges with other system devices; and a GUI manager 23 that allows to display a GUI remotely on a device having an interactive screen, for e.g., a tablet or smartphone.

The file called Device Shape contains a description of an interface function which is a provided capability when enabled (Interface Function 1) and a description of an interface function which is a consumed capability when in modified form (Interface Function 2).

### Description of the Capabilities Which Can Be Utilized by the Software Package of the Pump

As just mentioned, there are two such capabilities, respectively Interface Function 1 and Interface Function 2.

### Interface Function 1

When enabled, Interface Function 1 supplements the GUI with data provided by a paired system device, whatever the paired system device is.

This capability description means: the pump, as a capability consumer with graphic skills, is able to optionally display one and only one flow process value if the paired system device provides at least the process value, the process value name and unit using the OPC UA standard. No other restriction or condition is imposed for the negotiation or pairing except the maximum number of authorized pairing.

In such a variant, the capability description further means that the paired system devices may optionally provide the minimum and the maximum values of a range associated to the flow process value.

### Interface Function 2

When in original form, Interface Function 2 displays the pump motor speed and has a control icon allowing to start/stop the motor of the pump and a control icon allowing to set the pump motor speed. When in modified form, the two control icons are removed from the GUI, only the display of the pump motor speed is kept on display.

This capability description means: the pump 13, as a capability provider with pumping skills, is able to provide the control and monitoring on its pumping function using the OPC UA standard. The paired system device will have the exclusivity of the usage of the pumping function and will be able to start/stop the pump, to set the pump speed and to retrieve the current pumping status and speed. The OPC UA tag values for each of the control and monitoring are specified allowing the consumer to use the pump with an OPC UA client.

In such a variant, the capability description further means that the pump 13 will be able to provide the minimum and maximum values of a speed range.

### Discovery, Negotiation, Pairing (DNP)

A description will now be given on how system devices 13 and 14 can cooperate over the network 12 for establishing a paired condition.

This is mainly carried out by the DNP managers 19 and 25 in the system devices 13 and 14, through steps of discovery, negotiation and pairing, described below.

### Discovery

When connected to the network with IP (e.g. ethernet, Wi-Fi, Bluetooth or cellular such as 5G), the system device can see and can be seen by another connected system device that includes a discovery tool.

Several architectures already exist that enables discovery across a network (e.g. The ‘Bonjour’ protocol defined by Apple, and here global or local discovery proposed by the OPC UA standard).

The visibility is only restrained by potential security policies in place on the network

The discovery tool allows a system device to maintain an up-to-date list of visible system devices it can exchange information with. This list is notably updated when a new system device is connected to or disconnected from the network.

### Negotiation

Based on the capability descriptions provided in the Device Shape files, a negotiation starts between the connected system devices wherein each system device on the network: consults the capability descriptions exhibited by the other system devices, identifies matching capabilities based on the capability features Domain, Purpose and Role, verifies that it can respect the restrictions and conditions associated with the matching capabilities, and checks if the list of properties exhibited with the matching capabilities are the expected ones.

The Negotiation procedure occurs each time a new system device is discovered on the network, disconnected from the network, or no longer reachable.

### Pairing

Once the negotiation is achieved, the different contributors have agreed on how to adapt themselves to respect the restrictions and the conditions as expressed for each of the shared capabilities.

The pairing will complete the procedure, confirming the negotiation between two system devices.

The capability consumer (respectively the provider) memorizes the identification and location—here OPC UA endpoint—of the provider (respectively the consumer) to enable later data exchange.

Both the capability consumer and the capability provider apply restrictions and conditions—if some—agreed during the negotiation.

The capability consumer locally publishes the access to the list of properties in the Device Shape file of the consumed capability, so that the GUI manager installed in the capability consumer can exchange data with the paired capability provider.

This can be achieved, for example, using a standard Publication/Subscription approach: when the system device is powered on, a specific data queue is created by the DNP manager for each different pair (domain, purpose) described in the Device Shape file; the GUI manager subscribe to the (domain, purpose) queues it is interested in; each time a pairing occurs, the DNP manager publishes the access information in the corresponding (domain, purpose) data queue; the GUI manager subscriber to this queue will automatically be triggered and will access to the description and will accordingly adapt.

Specific situations could occur where different system devices on the network can provide a specific capability expected by a consumer. In such a case, the pairing might require a decision by a human operator to manually select the capability provider.

A similar situation requiring the approval from the operator occurs when this is explicitly expressed in the capability description as a condition.

It should be noted that the pairing removal requires the intervention of an operator to be able to distinguish between an intentional disconnection of a system device, and a communication failure.

Without this voluntary action from the user, any disconnection of a system device is considered as an anomaly and processed accordingly such as generating an alarm.

A description of pairing removal will be given now.

### Pairing Removal

As just mentioned, the pairing removal of a system device from another system device to which it is paired requires a voluntary and explicit action of the user, so as to enable to distinguish between an intentional disconnection of a system device and a communication failure.

This is carried out for instance by providing a dedicated menu accessible to the user on the graphical user interface of a system device, such menu listing each other system device with which the system device is paired, and from such menu the user can explicitly request to remove the pairing with a system device selected in the list of the menu.

When the user requests to remove the pairing with a selected other system device, steps are carried out (i) for removing the effects of the step of pairing, (ii) for removing the effects, if any, of the step of negotiation and (iii) for temporarily preventing the system device and the selected other system device to carry out a negotiation step.

For removing the effects of the step of pairing, the DNP manager 19 or 25 of the system device locally publishes the status change of each capability consumed from or provided to the selected other system device and sends to the selected other system device through the MtoM communication tool 18 or 24 a request to proceed to pairing removal. The DNP manager 19 or 25 of the selected other system device in turn locally publishes the status change of each capability consumed from or provided to the system device and sends to the system device through the MtoM communication tool 18 or 24 an acknowledgement receipt of the request to proceed to pairing removal.

In the system device and in the selected other system device, the GUI manager 17 or 23 is warned of the status change of each concerned capability and adapts accordingly.

For removing the effects of the step of negotiation, if any, namely if the capability consumer and the capability provider have applied restrictions and conditions agreed during the negotiation (e.g. exclusivity), these restrictions and conditions are invalidated.

For temporarily preventing the system device and the selected other system device to carry out a negotiation step, because the capabilities previously shared are now again available for negotiation, a quarantine is implemented for instance using a timeout such as not accepting the concerned system device in a negotiation step during a predetermined duration, the length of which is not really important but may, for example, be one minute, or 2 minutes, or 3 minutes, or 4 minutes, or 5 minutes, or 10 minutes, or using network connectivity such as ignoring the concerned system device until it is disconnected from the network and reconnected.

A detailed description will now be given of the pairing of the mixer and the pH sensor, of the pairing of the pump and the flow sensor, of the pairing of the previously paired mixer and pH sensor with the previously paired pump and flow sensor, and of the removal from the mixer of the paired pump and flow sensor while the pH sensor remains paired with the mixer.

### Pairing of the Mixer and the pH Sensor

### Features of the Mixer GUI

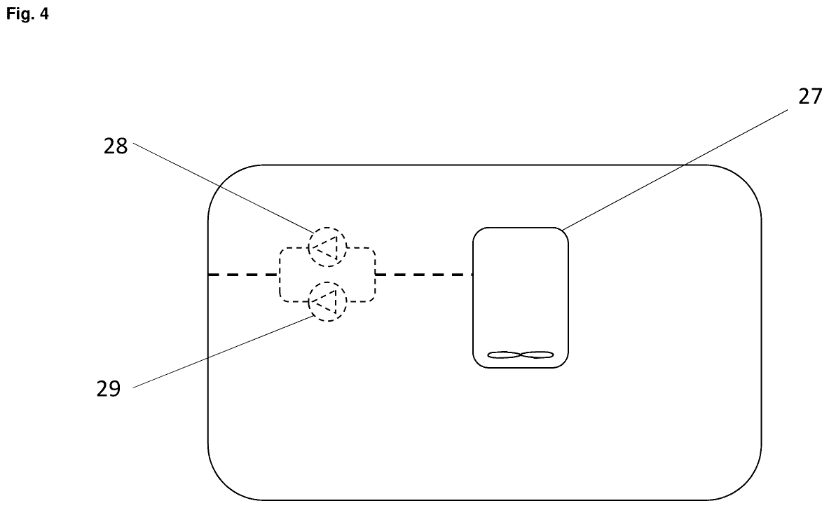

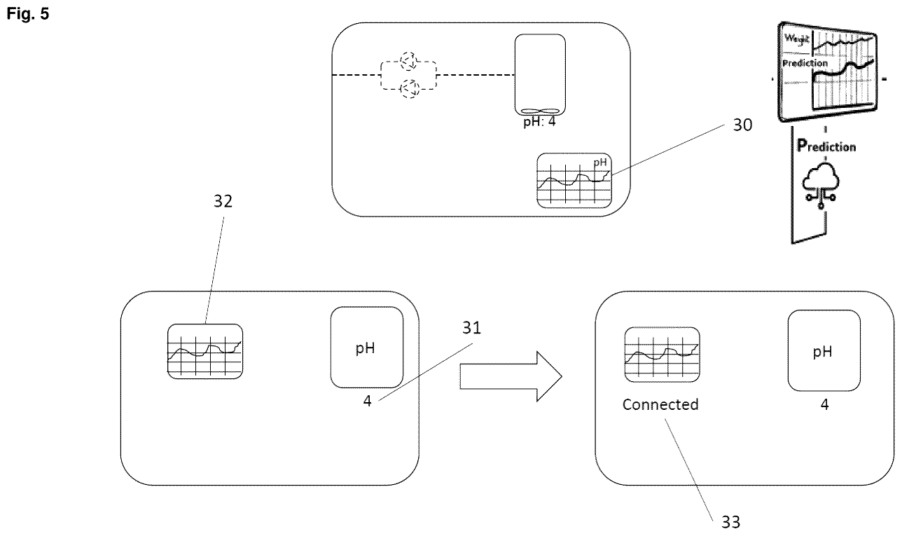

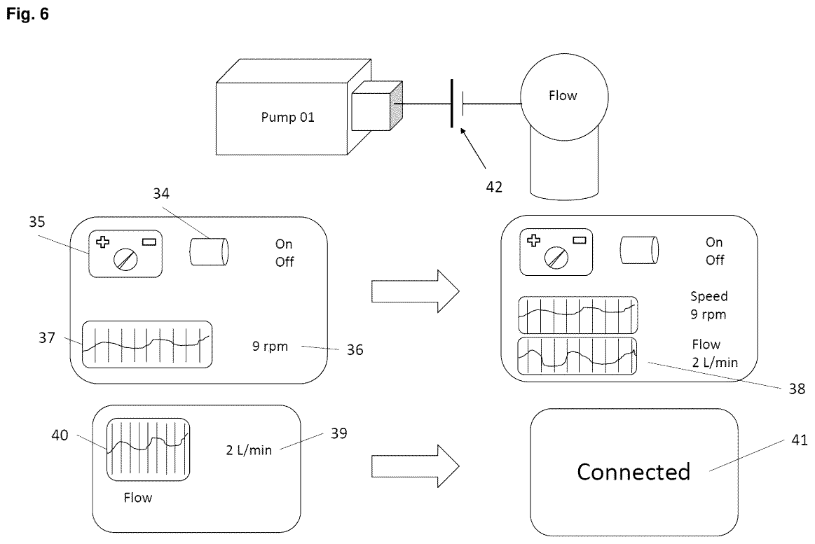

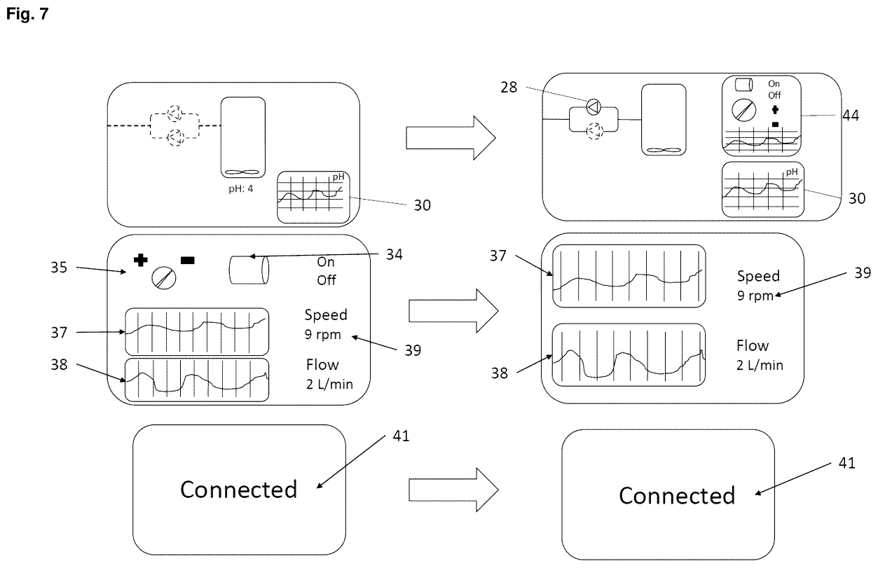

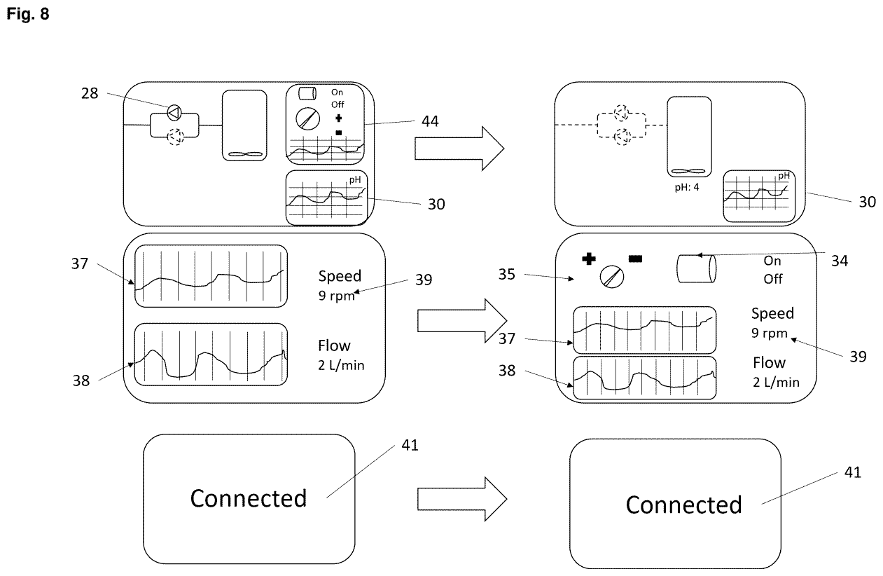

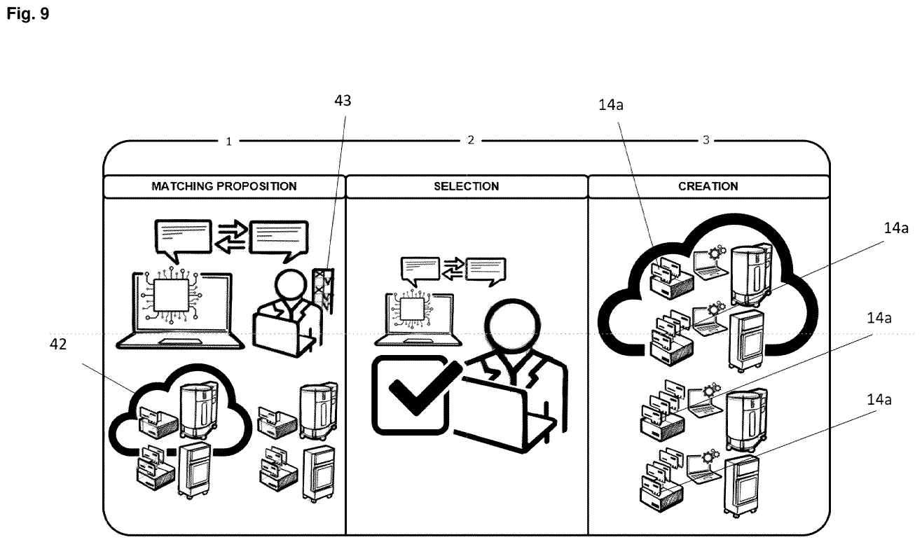

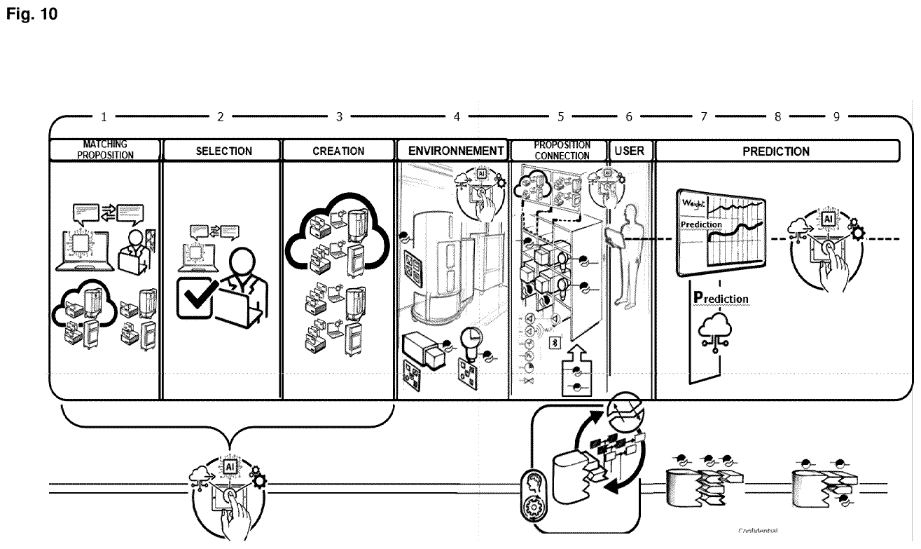

When the operator powers the mixer on, he can access to a basic P&ID GUI shown on FIG. 4, locally on an interactive screen or remotely on a device having an interactive screen, for e.g., a tablet or smartphone.

The basic P&ID GUI represents the different components required for the mixing process, an icon 27 representing a tank provided with an agitator, an icon 28 representing a mandatory pump in fluidic communication with inlet 1 of the tank and an icon 29 representing an optional pump in fluidic communication with inlet 2 of the tank.

In the basic P&ID GUI, icon 27 is displayed in a way showing that the tank provided with an agitator is present and operational (for instance displayed in permanent solid lines), icon 28 is displayed in a way showing that the mandatory pump is missing (for instance displayed in blinking phantom lines) and icon 29 is displayed in a way showing that the optional pump is missing (for instance displayed in permanent phantom lines).

The way the two icons 28 and 29 representing a pump are displayed is dependent on the pairing context and is automatically updated for showing that a corresponding pump system device is paired (for instance by then displaying the icon in permanent solid lines).

A further description of the P&ID GUI as regards the pairing with pumps will be given later in the section relating to the pairing of the mixer with the pump. Now will be given a description of the P&ID GUI as regards the pairing with the pH sensor.

When the mixer is powered on, its DNP manager creates in the digital controller of the mixer a (“Graphics”, “Process Value Display”) capability data queue. The GUI manager of the pump subscribes to the (“Graphics”, “Process Value Display”) capability data queue and displays the basic P&ID GUI until a new capability description is published in this queue.

When later a system device providing a (“Graphics”, “Process Value Display”) capability with the expected properties is paired with the mixer, the DNP manager of the mixer publishes the description of this capability in the (“Graphics”, “Process Value Display”) data queue, the GUI manager is triggered, accesses the published description and accordingly adapts the P&ID GUI, as shown on FIG. 5 at the top, by further displaying the current process value (here the pH) and a trend curve 30 for the process value.

Indeed, as described above, the Device Shape file of the mixer includes a capability named Interface Function 1 which, when enabled, supplements the P&ID GUI with process data provided by paired system devices, whatever the paired system devices are; and this capability has a description meaning: the mixer, as a capability consumer with graphic skills, is able to display a process value from several paired system devices if these paired system devices provide at least the process value, the process value name and unit and optionally the number of significant decimal digits using the OPC UA standard. No restriction or condition are imposed for the negotiation or pairing.

### Features of the pH Sensor GUI

When the operator powers the pH sensor on, he can access to an original GUI, shown on FIG. 5 at the bottom left, remotely on a device having an interactive screen, for e.g., a tablet or smartphone.

The original GUI includes the current value 31 measured by the pH sensor and a trend curve 32 representing the variation of the pH in time.

When the pH sensor is powered on, its DNP manager creates in the digital controller of the pH sensor a (“Graphics”, “Process Value Display”) capability data queue. The GUI manager of the pH sensor subscribes to the (“Graphics”, “Process Value Display”) capability data queue and displays the original GUI until a new capability description is published in this queue.

When later a system device consuming a (“Graphics”, “Process Value Display”) capability with the expected properties is paired with the pH sensor, the DNP manager of the pH sensor publishes the description of this capability in the (“Graphics”, “Process Value Display”) data queue, the GUI manager is triggered, accesses the published description and accordingly adapts the GUI, as shown on FIG. 5 at the bottom right, by additionally displaying the message “CONNECTED” 33.

Indeed, as described above, the Device Shape file of the pH sensor includes a capability which, when in modified form, keeps the display of the current value measured by the pH sensor and a trend curve representing the variation of the pH in time and adds an indication that the flow sensor is paired with an appropriate other system device, this indication being here the message “CONNECTED” 33; and this capability has a description meaning: the pH sensor, as a capability provider, is able to provide a pH (and only a pH) process value display dataset to paired system devices using the OPC UA standard. The dataset includes a process value, its name and its unit. The OPC UA tag values for each data item is specified allowing the paired system device to read these values with an OPC UA client. No specific restriction or condition is imposed for the negotiation or the pairing.

### DNP Sequence

The operator connects the mixer and the pH sensor to the same network 12.

DNP as described above is carried out and when done the P&ID GUI of the mixer and the GUI of the pH sensor are automatically updated: the P&ID GUI of the mixer is further displaying the current pH value and a trend curve 30 for the pH value; and the GUI of the pH sensor additionally displays a message “CONNECTED” 33.

It goes without saying that for enabling the pH sensor to sense the pH of the fluid being treated by the mixer, the pH sensor must be physically coupled to the mixer.

The message “CONNECTED” on the GUI of the pH sensor clearly shows that the pH sensor is not in stand-alone condition but in paired condition.

A detailed description of an example of DNP sequence will now be given.

In this example the pH sensor is first connected to the network 12 with IP, but of course the reverse is possible.

Since the pH sensor is a capability provider for the capability described in its Device Shape file 26, as long as the pH sensor is connected to the network 12, its MtoM communication tool 24 is provided in real time with the sensed pH value and makes it available on the network 12 thanks to the OPC UA server it includes, at the OPC UA endpoint given in the capability description in the Device Shape file 26, namely opc.tcp://pH/4:control/4:

For enabling DNP, in the pH sensor the DNP manager 25 provides the MtoM communication tool 24 with data to make available the capability description in the Device Shape file 26, including the properties in the capability description; and the DNP manager 25 creates in the digital controller 22 of the pH sensor a (“Graphics”, “Process Value Display”) data queue for this capability. The MtoM communication tool 24 then waits the discovery of another system device on the network 12.

Still in the pH sensor, for preparing to adapt, the GUI manager 23 subscribes to the (“Graphics”, “Process Value Display”) data queue created by the DNP manager 25 and displays the original form of the GUI.

The mixer in turn connects to the network and carries out similar steps in accordance with its Device Shape file 20, as detailed below.

For enabling DNP, in the mixer the DNP manager 19 provides the MtoM communication tool 18 with data to make available the capability descriptions in the Device Shape file 20, namely Interface Function 1, Interface Function 2, Interface Function 3 and Interface Function 4, including the properties in each capability description; and the DNP manager 19 creates in the digital controller 16 of the mixer a (“Graphics”, “Process Value Display”) data queue for the capability Interface Function 1 and a (“Control”, “Pumping”) data queue for the capabilities Interface Function 2, Interface Function 3 and Interface Function 4. The MtoM communication tool 18 then waits the discovery of another system device on the network.

Still in the mixer, for preparing to adapt, the GUI manager 17 subscribes to the (“Graphics”, “Process Value Display”) and (“Control”, “Pumping”) data queues created by the DNP manager and displays the basic P&ID GUI.

In the pH sensor, when the MtoM communication tool 24 discovers that the mixer is connected to the network 12, it informs of this discovery the DNP manager 25 which then requests the MtoM communication tool 24 to provide the capability descriptions exhibited by the mixer. When provided, the capability descriptions are reviewed by the DNP manager 25 which identifies a matching between the capability Interface Function 1 made available by the mixer and the local capability with the restrictions/conditions applicable. The DNP manager 25 then requests the MtoM communication tool 24 to propose to the mixer to apply pairing between the local capability and the capability Interface Function 1 in the mixer. When the MtoM communication tool 24 receives the pairing acceptance it provides the pairing acceptance to the DNP manager 25 which publishes the description of the capability Interface Function 1 of the mixer in the (“Graphics”, “Process Value Display”) data queue and requests the MtoM communication tool 24 to confirm application for the capability Interface Function 1 of the mixer. The GUI manager 23 is automatically notified of the publication in the (“Graphics”, “Process Value Display”) data queue and receives the description of the capability Interface Function 1 of the mixer and adapts accordingly, namely displays the modified form of the GUI, i.e. additionally displays the message “CONNECTED” 33. The modified form of the GUI is displayed until pairing removal occurs.

In the mixer, when the MtoM communication tool 18 discovers that the pH sensor is connected to the network 12, it informs of this discovery the DNP manager 19 which then requests the MtoM communication tool 18 to provide the capability descriptions exhibited by the pH sensor. When provided, the capability descriptions are reviewed by the DNP manager 19 which identifies a matching between the capability Interface Function 1 made available by the pH sensor and the local capability with the restrictions/conditions applicable. When the MtoM communication tool 18 receives from the pH sensor the confirmation of application for the capability Interface Function 1, the confirmation is transferred to the DNP manager 19 which publishes the description of the capability of the pH sensor in the (“Graphics”, “Process Value Display”) data queue. The GUI manager 17 is automatically notified of the publication in the (“Graphics”, “Process Value Display”) data queue and receives the description of the capability of the pH sensor, including the OPC UA tag for the flow value opc.tcp://pH/4:control/4:V, and adapts accordingly, namely adapts the P&ID GUI by further displaying the current pH value and a trend curve 30 for the pH value, the tag provided for the pH value being used, thanks to the OPC UA client in the MtoM communication tool 18, for continuously update the pH value until pairing removal occurs.

### Pairing of the Pump and the Flow Sensor

### Features of the Pump GUI

When the operator powers the pump on, he can access to a basic GUI, shown on FIG. 6 at the middle left, remotely on a device having an interactive screen, for e.g., a tablet or smartphone.

The basic P&ID GUI includes a Start/Stop button 34 allowing to operate the pump, a variator 35 allowing to modify the pump speed, a display of the current pump speed 36 and a display of a curve 37 representing the variation of the pump speed in time.

When the pump is powered on, its DNP manager 25 creates in the digital controller 22 of the pump a (“Graphics”, “Process Value Display”) capability data queue. The GUI manager 23 of the pump subscribes to the (“Graphics”, “Process Value Display”) capability data queue and displays the basic GUI until a new capability description is published in this queue.

When later a system device providing a (“Graphics”, “Process Value Display”) capability with the expected properties is paired with the pump, the DNP manager 25 of the pump publishes the description of this capability in the (“Graphics”, “Process Value Display”) data queue, the GUI manager 23 is triggered, accesses the published description and accordingly adapts the GUI, as shown on FIG. 6 at the middle right, by further displaying the current flow value and a trend curve 38 for the flow value.

Indeed, as described above, the Device Shape file of the pump includes a capability named Interface Function 1 which, when enabled, supplements the GUI with data provided by a paired system device; and this capability has a description meaning: the pump, as a capability consumer with graphic skills, is able to optionally display one and only one flow process value if the paired system device provides at least the process value, the process value name and unit using the OPC UA standard. No other restriction or condition is imposed for the negotiation or pairing except the maximum number of authorized pairing.

### Features of the Flow Sensor GUI

When the operator powers the flow sensor on, he can access to an original GUI, shown on FIG. 6 at the bottom left, remotely on a device having an interactive screen, for e.g., a tablet or smartphone.

The original GUI includes a display of the current value 39 measured by the flow sensor and a display of a trend curve 40 representing the variation of the flow in time.

When the flow sensor is powered on, its DNP manager 25 creates in the digital controller 22 of the flow sensor a (“Graphics”, “Process Value Display”) capability data queue. The GUI manager 23 of the flow sensor subscribes to the (“Graphics”, “Process Value Display”) capability data queue and displays the original GUI until a new capability description is published in this queue.

When later a system device consuming a (“Graphics”, “Process Value Display”) capability with the expected properties is paired with the flow sensor, the DNP manager 25 of the flow sensor publishes the description of this capability in the (“Graphics”, “Process Value Display”) data queue, the GUI manager 23 is triggered, accesses the published description and accordingly adapts the GUI, as shown on FIG. 6 at the bottom right, by only displaying the message “CONNECTED” 41.

Indeed, as described above, the Device Shape file 26 of the flow sensor includes a capability which, when in modified form, replaces the display of the current value measured by the flow sensor and the display of a trend curve representing the variation of the flow in time by the display of an indication that the flow sensor is paired with an appropriate other system device, this indication being here the message “CONNECTED” 41; and this capability has a description meaning: the flow sensor, as a capability provider, is able to provide a flow (and only a flow) process value display dataset to paired system devices using the OPC UA standard. The dataset includes a process value, its name and its unit. The OPC UA tag values for each of the data is specified allowing the paired system devices to read these values with an OPC UA client. No specific restriction or condition is imposed for the negotiation or the pairing.

### DNP Sequence

The operator connects the pump and the flow sensor to the same network 12 (FIG. 3).

DNP as described above is carried out and when done the GUI of the pump and the GUI of the flow sensor are automatically updated: the P&ID GUI of the pump is further displaying the current flow value and a trend curve 38 for the flow value; and the GUI of the flow sensor only displays a message “CONNECTED” 41.

It goes without saying that for enabling the flow sensor to sense the flow of the fluid driven by the pump, the flow sensor must be physically coupled in a known manner to the pump or to pipes in which flows the fluid driven by the pump, as shown on FIG. 6 at the top by reference numeral 42.

It should be noted that since the pump and the flow sensor are both a machine helper 14, the physical coupling 42 is between two treater helpers 21 (and not between a fluid treater 15 and a treater helper 21).

It should be further noted that the DNP manager 25 of the pump is able to behave as the DNP manager 19 of a bioprocess machine 13 with respect to the DNP manager 25 of the flow sensor for cooperating over the network 12 for establishing a paired condition between the pump and the flow sensor, thanks to the fact that the capability Interface Function 1 of the pump has as Role feature “Consumer”, just as each capability in the Device Shape file 20 of a bioprocess machine 13.

The message “CONNECTED” 41 on the GUI of the flow sensor clearly shows that the flow sensor is not in stand-alone condition but in paired condition.

A detailed description of an example of DNP sequence will now be given.

In this example the flow sensor is first connected to the network 12 with IP, but of course the reverse is possible.

Since the flow sensor is a capability provider for the capability described in its Device Shape file 26, as long as the flow sensor is connected to the network 12, its MtoM communication tool 24 is provided in real time with the sensed flow value and makes it available on the network 12 thanks to the OPC UA server it includes, at the OPC UA endpoint given in the capability description in the Device Shape file 26, namely opc.tcp://flow/4:control/4:

For enabling DNP, in the flow sensor the DNP manager 25 provides the MtoM communication tool 24 with data to make available the capability description in the Device Shape file 26, including the properties in the capability description; and the DNP manager 25 creates in the digital controller 22 of the flow sensor a (“Graphics”, “Process Value Display”) data queue for this capability. The MtoM communication tool 24 then waits the discovery of another system device on the network 12.

Still in the flow sensor, for preparing to adapt, the GUI manager 23 subscribes to the (“Graphics”, “Process Value Display”) data queue created by the DNP manager 25 and displays the original form of the GUI.

The pump in turn connects to the network and carries out similar steps in accordance with its device Shape file 26, as detailed below.

As regards the capability Interface Function 2 (for which the pump is a capability provider), as long as the pump is connected to the network 12, its MtoM communication tool 24 is provided in real time with the operating parameters of the pump (Start/Stop control, started status, speed setting and value) and makes it available on the network 12 thanks to the OPC UA server it includes, at the OPC UA endpoints given in the capability description in the Device Shape file 26, respectively opc.tcp://start, opc.tcp://started and opc.tcp://speed/4:

For enabling DNP, in the pump the DNP manager 25 provides the MtoM communication tool 24 with data to make available the capability descriptions in the Device Shape file 26, namely Interface Function 1 and Interface Function 2, including the properties in each capability description; and the DNP manager 25 creates in the digital controller 22 of the pump a (“Graphics”, “Process Value Display”) data queue for the capability Interface Function 1 and a (“Control”, “Pumping”) data queue for the capability Interface Function 2. The MtoM communication tool 24 then waits the discovery of another system device on the network 12.

Still in the pump, for preparing to adapt, the GUI manager 23 subscribes to the (“Graphics”, “Process Value Display”) and (“Control”, “Pumping”) data queues created by the DNP manager 25 and displays the basic GUI.

In the flow sensor, when the MtoM communication tool 24 discovers that the pump is connected to the network 12, it informs of this discovery the DNP manager 25 which then requests the MtoM communication tool 24 to provide the capability descriptions exhibited by the pump. When provided, the capability descriptions are reviewed by the DNP manager 25 which identifies a matching between the capability Interface Function 1 exhibited by the pump and the local capability with the restrictions/conditions applicable. The DNP manager 25 then requests the MtoM communication tool 24 to propose to the pump to apply pairing between the local capability and the capability Interface Function 1 in the pump. When the MtoM communication tool 24 receives the pairing acceptance it provides the pairing acceptance to the DNP manager 25 which publishes the description of the capability Interface Function 1 of the pump in the (“Graphics”, “Process Value Display”) data queue and requests the MtoM communication tool 24 to confirm application for the capability Interface Function 1 of the pump. The GUI manager 23 is automatically notified of the publication in the (“Graphics”, “Process Value Display”) data queue and receives the description of the capability Interface Function 1 of the pump and adapts accordingly, namely displays the modified form of the GUI, i.e. only displays the message “CONNECTED” 41. The modified form of the GUI is displayed until pairing removal occurs.

In the pump, when the MtoM communication tool 24 discovers that the flow sensor is connected to the network 12, it informs of this discovery the DNP manager 25 which then requests the MtoM communication tool 24 to provide the capability descriptions exhibited by the flow sensor. When provided, the capability descriptions are reviewed by the DNP manager 25 which identifies a matching between the capability Interface Function 1 made available by the flow sensor and the local capability with the restrictions/conditions applicable. When the MtoM communication tool 24 receives from the flow sensor the confirmation of application for the capability Interface Function 1, the confirmation is transferred to the DNP manager 25 which publishes the description of the capability of the flow sensor in the (“Graphics”, “Process Value Display”) data queue. The GUI manager 23 is automatically notified of the publication in the (“Graphics”, “Process Value Display”) data queue and receives the description of the capability of the flow sensor, including the OPC UA tag for the flow value opc.tcp://flow/4:control/4:V, and adapts accordingly, namely adapts the GUI as shown on FIG. 6 at the middle right by further displaying the current flow value and a trend curve 38 for the flow value, the tag provided for the flow value being used, thanks to the OPC UA client in the MtoM communication tool 24, for continuously update the flow value until pairing removal occurs.

It should be noted that there is in the Device Shape file 26 of the pump a further capability enabling the pump to provide the flow values sensed by the flow sensor as if the flow sensor was part of the pump.

This will be described later. At the moment, it suffices to note that this enables the trend curve of the flow to be included in the control panel 44 in the P&ID GUI of the mixer, as shown at the top of FIGS. 7 and 8.

### Pairing of the Previously Paired Mixer and pH Sensor with the Previously Paired Pump and Flow Sensor

### Further Features of the Mixer GUI

As described above, upon pairing with the pH sensor the P&ID GUI of the mixer is supplemented, with respect to the basic P&ID, with a display of the pH value and a display of a trend curve for the pH value, as shown on FIG. 5 at the top.

As described above, the basic P&ID GUI, shown on FIG. 4, has an icon 27 representing a tank provided with an agitator, an icon 28 representing a mandatory pump in fluidic communication with inlet 1 of the tank and an icon 29 representing an optional pump in fluidic communication with inlet 2 of the tank; icon 27 is displayed in a way showing that the tank provided with an agitator is present and operational (for instance displayed in permanent solid lines), icon 28 is displayed in a way showing that the mandatory pump is missing (for instance displayed in blinking phantom lines) and icon 29 is displayed in a way showing that the optional pump is missing (for instance displayed in permanent phantom lines). The way the two icons 28 and 29 representing a pump are displayed is dependent on the pairing context and is automatically updated for showing that a corresponding pump system device is paired (for instance by then displaying the icon in permanent solid lines).

Further details as regards the pairing with the pH sensor are given elsewhere in the present description.

Further details will be given now as regards the pairing with pumps.

When the mixer is powered on, its DNP manager 19 creates in the digital controller 16 of the mixer a (“Control”, “Pumping”) capability data queue. The GUI manager 17 of the mixer subscribes to the (“Control”, “Pumping”) capability data queue. The icon 28 is displayed in a way showing that the mandatory pump is missing (for instance displayed in blinking phantom lines) until a new capability description is published in this queue with a Control_Local_Name equal to “Pump_on_inlet1”. The icon 29 is displayed in a way showing that the optional pump is missing (for instance displayed in permanent phantom lines) until a new capability description is published in this queue with a Control_Local_Name equal to “Pump_on_inlet2”.

When later a system device providing a (“Control”, “Pumping”) capability is paired with the mixer, the DNP manager 19 of the mixer publishes the description of this capability completed with Control_Local_Name equal to “Pump_on_inlet1” in the (“Control”, “Pumping”) capability data queue, the GUI manager 17 is triggered, accesses the published description and accordingly adapts the P&ID GUI by displaying the icon 28 in a way showing that the mandatory pump is paired (for instance displayed in permanent solid lines), as illustrated on FIG. 7 at the top right.

Indeed, as described above, the Device Shape file 20 of the mixer includes a capability named Interface Function 2 which, when enabled, adapts the P&ID GUI to show if the mandatory expected pump has been paired and supplements the P&ID GUI with control icons and displays of operating parameters of the paired pump; and this capability has a description meaning: the mixer, as a capability consumer with control skills, requires a system device which is a pump to be mandatorily paired to achieve the role defined for the pump connected to the inlet 1. To be paired, the system device will mandatorily have to make available a Start/Stop command and provide its current started status. The mixer will be able to control and monitor the pump speed if provided by the paired system device. The confirmation by the operator is required during the pairing procedure. Once paired, the mixer will have the exclusive usage of the system device which is a pump.

Similarly, when later a system device providing a (“Control”, “Pumping”) capability is paired with the mixer, the DNP manager 19 of the mixer publishes the description of this capability completed with Control_Local_Name equal to “Pump_on_inlet2” in the (“Control”, “Pumping”) capability data queue, the GUI manager 17 is triggered, accesses the published description and accordingly adapts the P&ID GUI by displaying the icon 29 in a way showing that the optional pump is paired (for instance displayed in permanent solid lines).

Indeed, as described above, the Device Shape file 20 of the mixer includes a capability named Interface Function 3 which, when enabled, adapts the P&ID GUI to show if the planed optional pump has been paired and supplements the P&ID GUI with control icons and displays of operating parameters of the paired pump; and this capability has a description meaning: the mixer, as a capability consumer with control skills, is able to control an optional system device which is a pump to achieve the predefined role for the pump connected to the inlet 2. To be paired, the system device will mandatorily have to make available a Start/Stop command and provide its current started status. The mixer will be able to control and monitor the pump speed if provided by the paired system device. The confirmation by the operator is required during the pairing procedure. Once paired, the mixer will have the exclusive usage of the system device which is a pump.

### Further Features of the Pump GUI

The operator now connects the pump on the same network 12.

When the mixer discovers the pump, the P&ID GUI of the mixer warns the operator (with a non-illustrated display, for instance in a pop-up window) that a pump that fulfills the requirements expected for a pump is available and can be used, and request to select one of the possible pumps.

Once the operator has physically connected the pump on the inlet 1 of the mixer, the choice inlet 1 may be selected.

The GUI of the flow sensor remains unchanged, that is to say still displays the message 41, as shown on FIG. 7 at the bottom.

The P&ID GUI of the mixer and the GUI of the pump are automatically updated.

In the P&ID GUI of the mixer, as shown on FIG. 7 at the top, icon 28 is displayed in a way showing that the mandatory pump on the inlet 1 is present and operational (for instance displayed in permanent solid lines), and a control panel 44 is now present on the P&ID GUI of the mixer, allowing the operator to control and monitor the pump on the inlet 1. The control panel 44 includes a Start/Stop button allowing to operate the pump, a variator allowing to modify the pump speed and a display of a trend curve representing the variation of the flow measured by the flow meter paired with the pump.

In the GUI of the pump, as shown on FIG. 7 at the middle, the Start/Stop button 34 allowing to operate the pump and the variator 35 allowing to modify the pump speed are removed. Only the display of the flow and the speed are kept.

When the pump is powered on, its DNP manager 25 creates in the digital controller 22 of the pump a (“Control”, “Pumping”) capability data queue. The GUI manager 23 of the pump subscribes to the (“Control”, “Pumping”) capability data queue and displays the basic GUI until a new capability description is published in this queue.

When later a system device providing a (“Control”, “Pumping”) capability with the expected properties is paired with the pump, the DNP manager 25 of the pump publishes the description of this capability in the (“Control”, “Pumping”) capability data queue, the GUI manager 23 is triggered, accesses the published description and accordingly adapts the GUI by removing the Start/Stop button 34 allowing to operate the pump and the variator 35 allowing to modify the pump speed.

Indeed, as described above, the Device Shape file 26 of the pump includes a capability named Interface Function 2 which, when in original form, displays the pump motor speed and has a control icon (button 34) allowing to start/stop the motor of the pump and a control icon (variator 35) allowing to set the pump motor speed. When in modified form, the two control icons are removed from the GUI, only the display of the pump motor speed is kept on display; and this capability has a description meaning: the pump, as a capability provider with pumping skills, is able to provide the control and monitoring on its pumping function using the OPC UA standard. The paired system device will have the exclusivity of the usage of the pumping function and will be able to start/stop the pump, to set the pump speed and to retrieve the current started status and speed. The OPC UA tag values for each of the control and monitoring are specified allowing the consumer to use the pump with an OPC UA client.

### DNP Sequence

The mixer (previously paired with the pH sensor) and the pump (previously paired with the flow sensor) are deployed on the same network 12, so that they can see each other and start the negotiation step.

The pump makes available the capability Interface Function 2.

The Mixer makes available three capabilities with the same domain and purpose (“Control”, “Pumping”): the capability Interface Function 2 requiring a pumping system to be mandatorily paired to achieve the role defined for the pump connected to the inlet 1; the capability Interface Function 3 enabling the mixer to control and monitor an optional pumping system to achieve the role defined for the pump connected to the inlet 2; and the capability Interface Function 4 enabling the mixer to control and monitor other optional pumping systems.

All three capabilities are matching with the capability exhibited by the pump.

The negotiation is successful, allowing to start the pairing procedure.

One restriction/condition is defined for the capability exhibited by the pump: “only one system can be paired with the pump to consume this capability”. As no system has yet been paired with the pump to consume this capability, this condition is verified and the pairing can occur.

One restriction/condition is defined for two of the three capabilities exhibited by the mixer: “the confirmation by the operator is required during the pairing”. The pairing will then only be achieved once confirmed by the operator.

A warning message is then displayed by the P&ID GUI of the mixer (not illustrated, for instance on a pop-up window), requesting the operator to assign the pump to one of the three possible usage.

Once the operator has assigned the pump to the inlet 1, the pairing procedure continues.

Both the mixer and the pump memorize the identification and location-OPC UA endpoint-of the paired system for this capability. In the drawn example, only the mixer will use this information to later control and monitor the pump. Once paired, this also avoids the pump from pairing with another system with this capability, too.

On both the mixer and the pump, the DNP manager 19 or 25 publishes the capability description in the (“Control”, “Pumping”) capability data queue. To conform to the selection done by the operator, on the mixer side, this capability is published with the “control application” value set to “Pump_on_inlet1”.

As described above, on both the mixer and the pump, the GUI manager 17 or 23 has subscribed to that capability data queue and automatically updates.

In a non-illustrated variant, instead of removing from the GUI of the pump the Start/Stop button 34 and the variator 35 upon pairing of the pump with an appropriate other system device such as the mixer, only the variator 35 is removed from the GUI of the pump, the Start/Stop button 34 being kept.

### Removal from the Mixer of the Paired Pump and Flow Sensor While the pH Sensor remains paired with the mixer

As mentioned above, the pairing removal of a system device from another system device to which it is paired requires a voluntary and explicit action of the user, so as to enable to distinguish between an intentional disconnection of a system device and a communication failure.

This is carried out here by providing a dedicated menu (not illustrated on the drawings) accessible to the user on the GUI of each system device, so that in the present example each of the P&ID GUI of the mixer, the GUI of the pH sensor, the GUI of the pump and the GUI of the flow sensor has such a dedicated menu.

This dedicated menu lists each other system device with which the system device is paired, and from such menu the user can explicitly request to remove the pairing with a system device selected in the list of the menu.

When the user requests to remove the pairing with a selected other system device, steps are carried out (i) for removing the effects of the step of pairing, (ii) for removing the effects, if any, of the step of negotiation and (iii) for temporarily preventing the system device and the selected other system device to carry out a negotiation step.

For removing the effects of the step of pairing, the DNP manager 19 or 25 of the system device locally publishes the status change of each capability consumed from or provided to the selected other system device and sends to the selected other system device through the MtoM communication tool 18 or 24 a request to proceed to pairing removal. The DNP manager 19 or 25 of the selected other system device in turn locally publishes the status change of each capability consumed from or provided to the system device and sends to the system device through the MtoM communication tool 18 or 24 an acknowledgement receipt of the request to proceed to pairing removal.

In the system device and in the selected other system device, the GUI manager 17 or 23 is warned of the status change of each concerned capability and adapts accordingly.

FIG. 8 illustrates the changes in the P&ID GUI of the mixer, in the GUI of the pump and in the GUI of the flow sensor further to the selection by the user, in the dedicated menu of the mixer, of the pump paired with the flow sensor as to be removed from pairing with the mixer.

Within the mixer, the DNP manager 19 publishes in the appropriate queue, namely the (“Control”, “Pumping”) queue, the status change of the corresponding capability, namely Interface Function 2, the GUI manager 17 is triggered and adapts accordingly by disabling the capability Interface Function 2 so that the control panel 44 is removed from the P&ID GUI of the mixer, as shown at the top of FIG. 8.

Still within the mixer, the DNP manager 19 requests the MtoM communication tool 18 to send to the pump a request to proceed to pairing removal.

This request is transmitted through the network 12, received by the MtoM communication tool 24 of the pump and transferred to the DNP manager 23 of the pump which then publishes in the appropriate queue, namely the (“Control”, “Pumping”) queue, the status change of the corresponding capability, namely Interface Function 2, the GUI manager 23 is triggered and adapts accordingly by putting in original form the capability Interface Function 2 so that the variator 35 and the Start/Stop button 34 become present on the GUI of the pump, as shown at the middle of FIG. 8.

As the flow sensor is not concerned by the pairing removal (it is still paired with the pump), no action is taken thereabout and accordingly its GUI remains unchanged, as shown on FIG. 8 at the bottom. This is the same for the pH sensor which is also not concerned by the pairing removal (it is still paired with the mixer).

The DNP manager 25 of the pump sends to the mixer through the MtoM communication tool 24 an acknowledgement receipt of the request to proceed to pairing removal.

As mentioned above, the effects of the step of negotiation between the mixer and the pump (exclusivity) are invalidated; and for temporarily preventing the mixer and the pump to carry out a negotiation step, because the capabilities previously shared are now again available for negotiation, a quarantine as described earlier is implemented.

The other pairing removals (mixer and pH sensor; pump and flow sensor) are carried out similarly.

### Capability Propagation

As mentioned above, there is in the pump a further capability enabling the pump to provide the flow values sensed by the flow sensor as if the flow sensor was part of the pump.

This further capability, named Interface Function 3, is described in the Device Shape file 26 of the pump.

The description of the capability Interface Function 3 includes an identifier (capabilityUniqueID) and also refers to the identifier (refToCapabilityUniqueID) of another capability, namely the relevant capability of the flow sensor paired with the pump, if any.

It should be noted in this respect that for simplifying the above disclosure, it was not mentioned above that the description of each capability includes an identifier (capabilityUniqueID) which is unique to the capability, in the present example a four-digit value.

### Interface Function 3

When available, Interface Function 3 is similar to the above disclosed interface function in the pH sensor: when in modified form Interface Function 3 of the pump keeps the display of the current value measured by the flow sensor and a trend curve representing the variation of the flow in time while the display is modified for having an indication that the pump is paired with an appropriate other system device, this indication being here the removal of the two control icons 34 and 35 from the GUI of the pump done by Interface Function 2 upon pairing with an appropriate other system device, such as the mixer.

The description of the capability Interface Function 3 of the pump means: the pump, as a capability provider, is able to provide a flow (and only a flow) Process Value Display dataset to paired system devices using the OPC UA standard. This capability will only be negotiable when the pump will have let it available. The dataset includes a process value, its name and its unit.

Generally speaking, a capability such as Interface Function 3, behaving when activated in the same way as a capability in a paired other system device and as if it was from the system device, can be provided in system devices other than a pump for paired system devices other than a flow sensor.

For mere language convenience, the mechanism involving a capability such as Interface Function 3 of the pump can be termed as capability propagation, with reference to the fact that the source capability (such as the capability in the flow sensor) is rendered available with the same substance through a system device such as the pump, able to activate a capability (such as Interface Function 3) replicating the source capability; and a capability such as Interface Function 3 can be termed a capability propagator.

Of course, since such a capability (such as Interface Function 3) replicates the source capability, the provided capability in the bioprocess machine 13 (here the mixer) replicates the provided capability in the concerned machine helper 14 (here the pump).

### Capability Multiple Sharing

In a variant not illustrated, the pump does not include a capability as Interface Function 3: the flow sensor, instead of being paired only with the pump, is paired together with the pump and with the mixer, so that the mixer gets the flow values directly from the flow sensor (and not from the pump which gets the flow values from the flow sensor).

Indeed, in the capability of the flow sensor, there is no restriction as to the number of other system device with which pairing is authorized while the capability Interface Function 1 of the mixer is able to display process value from several paired system devices, such as the flow sensor in addition to the pH sensor.

For mere language convenience, the fact that a capability (such as the capability in the flow sensor) is consumed by more than one other system device can be termed as capability multiple sharing.

### Capability Hierarchization

In another variant not illustrated, the capability Interface Function 3 is not merely replicating the source capability (the capability in the flow sensor) but enables the pump to provide an additional function the pump is not able to provide without the flow sensor, namely flow regulation.

### Interface Function 3

When available, Interface Function 3 is able to provide controlling and monitoring of the pump speed so as to regulate the flow through the pump using the OPC UA standard.

The description of the capability Interface Function 3 of the pump means: the pump, as a capability provider with Process Value Regulation skills is able to provide controlling and monitoring of the pump speed so as to regulate the flow through the pump using the OPC UA standard. This capability will only be negotiable when the pump will have let it available. The paired system device, such as the mixer, will then have the exclusivity of the control and will be able to Start/Stop the regulation, to set the regulation parameters and to retrieve data from the process value.

Generally speaking, a capability such as Interface Function 3, providing when activated an additional function, can be provided in system devices other than a pump for paired system devices other than a flow sensor.

For mere language convenience, the mechanism involving a capability such as Interface Function 3 of the pump can be termed as capability hierarchization, with reference to the fact that the source capability (such as the capability in the flow sensor) is embedded in a more complex capability; and a capability such as Interface Function 3 can be termed a hierarchized capability.

Though such a capability (such as Interface Function 3) may include the source capability, the provided capability in the bioprocess machine 13 (here the mixer) need to necessarily embed the provided capability in the concerned machine helper 14 (here the pump).

As used herein, the term “hierarchical capability” is generally used to denote that a source capability is mandatory for a machine helper to be able to expose/show the hierarchical capability. For example, “volume calculation” as hierarchical capability would, for example and depending on the shape of the volume to be determined, require “length”, “height” and “depth” as source capabilities.

### Further Variants

In variants to the examples disclosed above:
the bioprocess machine is different from a mixer, for instance a bioreactor, a chromatograph, a virus inactivation or a tangential flow filtration;
the machine helpers are different from a pump, a flow sensor and a pH sensor, for example other active components such as valves or mass flow controllers; other sensors whether mechanical, electronic, opto-electronic, infra-red, ultra-violet, etc. like pressure sensors, temperature sensors, OD (Optical Density) sensors, DO (Dissolved Oxygen) sensors, gas (CO2, . . . ) sensors, weight sensors, speed (RPM . . . ) sensors, flow (gas/air) rate sensors, humidity sensors, hygrometry sensors, light/lux sensors, position (valve, actuator, switch . . . ) sensors, power (watt . . . ) sensors, galvanometers, motion sensors, vacuum sensors, title sensors, viability sensors, resistivity sensors, proximity/distance sensors, volume sensors, UV sensors, IR sensors, frequency sensors, molar concentration sensors, duration/time sensors, radiation sensors, colorimeters, glucometers, opacimeters, osmometers, photometers, spectroscopes, acoustic pressure sensors, sonometers, video sensors, photo sensors, electrical charge sensors, particle counters, viscosity sensors or lactate sensors; other types of instrumentation; and/or ancillary devices such as a cell retention device or a mixer which are capability providers (unlike the mixer in the above example);
unlike the mixer in the above example for which it is mandatory to be paired with a pump connected to inlet 1 for being operable, the bioprocess machine is operable in stand-alone condition, that is to say without having to be paired to a bioprocess machine helper for being operable;
the system devices are originally in places different from a production area and a storage area, for instance all system devices are originally in a storage area and they are all brought in a production area for setting up the installation;
the interactive screen is replaced or complemented by another user interface such as a passive display and physical buttons or a passive screen and a keyboard;
the network is different from a network with IP;
the machine to machine communication standard is different from OPC UA; and/or
in the system there is only one bioprocess machine and one bioprocess machine helper; or there is a plurality of bioprocess machines and a plurality of bioprocess machine helpers with at least certain machine helpers which can be paired with different bioprocess machines.

Many other variants are possible and it is recalled in this respect that the invention is not limited to the examples disclosed and illustrated.

One important preferred embodiment is, that the system can comprise virtual, software based components, which can be used to predict or compute the behaviour of the biotechnological fluid in the fluid treater using physico-chemical or biological properties and/or external data. These software based components then analyse the results and with the so gained information the capabilities of the whole system can be improved. FIG. 9 shows the necessary steps for adding such a virtual, software based component.

The following working example including FIG. 10 shows an preferred approach how to create and use a system including those software based components:

1. A lab scientist needs a DO (dissolved oxygen) prediction on his bioprocess. To acquire this DO prediction he uses a software running on a computer. This computer can be any kind of computing device, from a standard personal computer, a μ-Controller up until to a quantum computer, whatever is suitable. It can be also a local computer or a remote computer, like a cliud based computer. The software comprises of several modules with specific functions and contains information about the current bioprocess for which the lab scientist needs the DO prediction. One of those modules is a match helper which shows the lab scientist the available helpers which are adapted to and can be used with the assigned bioprocess.

2. The lab scientist then selects a suitable DO prediction function in the match helper module and this information is then communicated to the adaptative device creator. This device creator is another software module which creates the virtual component, in this case a DO prediction helper.

3. With the given information the adaptative device creator then creates a virtual new DO prediction helper in a smart component in the lab which is then virtually added to the bioprocess machine system.

4. From a storage or production area a suitable mixer as defined in an available process orchestration recipe is then selected as a physical component and added to the system. The computer which hosts the virtual DO prediction helper, e.g. the personal computer or any other suitable computer, also contains all required data about the physical system and its components, which enables it and the virtual, software based DO prediction helper to perform its prediction. When all planned components are added to the system it is powered on. The software also provides then a basic P&ID about the installed system.

5. The storage areas of all smart components, which can be either physical like the mixer or a pump or software based like the DO prediction helper are then installed.

6. The virtual DO prediction helper then computes the DO prediction data with the given data provided by the software.

7. The lab scientist then logs in to the system via a suitable machine interface (HMI) to access the reporting module for each smart component.

8. The wanted DO prediction data for the given system is then delivered to the lab scientist by the respective smart component which is available via its corresponding reporting module.

9. With this DO prediction data the lab scientist is then enabled to improve the system to result in a better dissolved oxygen level, without the need to perform a real test with physical components.

It is understood that while the virtual, software based components 14a are preferably adapted to be used with the disclosed system for treating a biotechnological fluid disclosed in this document the invention of creating and using such software based components are not limited to this system. The invention can be used for any comparable system with system components which are able to be simulated and used in the disclosed way.

1. A system for treating a biotechnological fluid, comprising
a bioprocess machine (13) having: a biotechnological fluid treater (15) which is configured for modifying at least one physico-chemical or biological property of said biotechnological fluid and a digital controller (16) for controlling said physical biotechnological fluid treater (15); and
at least one physical bioprocess machine helper (14) having: a physical biotechnological fluid treater helper (21) configured for being coupled to said biotechnological fluid treater (15) and a digital controller (22) for controlling said biotechnological fluid treater helpers (21); and/or
at least one software-based bioprocess machine helper (14a) created by an adaptive device creator (42); and
a software-based bioprocess machine match helper (43) for selecting a suitable software-based bioprocess machine helper (14a)
wherein:
said digital controller (16) of said bioprocess machine (13) and said digital controller (22) of said machine helper (14) each include at least one capability manager (17, 23), a machine to machine communication tool (18, 24) (MtoM communication tool), and a discovery negotiation pairing manager (19, 25) (DNP manager);
each said MtoM communication tool (18, 24) is configured for connecting to a network (12);
the DNP manager (19) of said bioprocess machine (13) and the DNP manager (25) of said machine helpers (14) are configured for cooperating over said network (12) for establishing a paired condition; wherein the capability manager (17) of the bioprocess machine (13) and the capability manager (23) of the machine helpers (14) are configured such that in said paired condition they organize the provision and/or consummation of the respective capabilities by either said bioprocess machine (13) or said machine helpers (14);
said software-based bioprocess machine helper (14a) is configured by said suitable adaptive device creator (42) to predict or compute the behaviour of said biotechnological fluid using physico-chemical or biological properties and/or external data, analyse the result and extend the capabilities of said physical bioprocess machine (13).

2. The system according to claim 1, wherein said adaptative device creator (42) and/or at least one software-based bioprocess machine helper (14a) is hosted either by a remote computer which transfers said created software-based bioprocess machine helper (14a) to its respective digital controller (16) or is hosted directly as a local instance on said respective digital controller (16).

3. The system according to claim 1, wherein said at least one software-based bioprocess machine helper (14a) is configured to have the same behaviour as its physical counterpart (14).

4. The system according to claim 1, wherein said digital controller (16) of said bioprocess machine (13) includes a file (20) containing a description of each said interface function which can be a said provided capability and said digital controller (22) of said machine helper (14) includes a file (26) containing a description of each said interface function which can be a said consumed capability.

5. The system according to claim 1, wherein the DNP manager (19) of said bioprocess machine (13) and the DNP manager (25) of said machine helper (14) are configured for cooperating over said network (12) for establishing a paired condition.

6. The system according to claim 1, wherein said system devices include a plurality of said machine helpers (14) and the DNP manager (19) of said bioprocess machine (13) is configured for establishing a said paired condition simultaneously with at least one said machine helper (14).

7. The system according to claim 1, wherein said system devices include a first said bioprocess machine (13), a second said bioprocess machine (13) and a plurality of said machine helpers (14); and the DNP manager (25) of at least one said machine helper (14) is configured for establishing a said paired condition either with said first bioprocess machine (13) or with said second bioprocess machine (13).

8. The system according to claim 1, having a first said machine helper (14) in which the consumed capability is an interface function controlling and/or displaying an operating parameter of the treater helper (21) of said first machine helper (14); and having a second said machine helper (14) in which the consumed capability is an interface function displaying a physico-chemical or biological quantity of the fluid sensed by the treater helper (21) of second machine helper (14), wherein the first machine helper (14) is a pump in which the consumed capability is an interface function controlling and/or displaying a speed of the pump and the second machine helper (14) is a pH sensor or a flow sensor in which the consumed capability is an interface function displaying a pH of said fluid or a flow of said fluid.

9. The system according to claim 1, wherein each said MtoM communication tool (18, 24) is configured for connecting to a said network (12) which is a network with Internet Protocol, such as Ethernet, Wi-Fi, Bluetooth or cellular 5G.

10. A method for operating a system for treating a biotechnological fluid according to claim 1, the method comprising:
Identifying relevant process context and required capabilities of said physical biotechnological fluid treater (15) and use it to select at least one suitable software-based bioprocess machine helper (14a) via said software-based bioprocess machine match helper (43)
creating the selected at least one software-based bioprocess machine helper (14a) via said adaptative device creator
adding all software-based devices (14a) to the system and pairing the at least one software-based bioprocess machine helper (14a) with the respective bioprocess machine (13) via the DNP manager (19);
predicting and/or computing the behavior of said biotechnological fluid in said physical biotechnological fluid treater (15) using its at least one physico-chemical or biological properties and/or external data via said software-based bioprocess machine helper (14a);
analyzing the data and with the results adapting the configuration of said physical biotechnological fluid treater (15) and extending the capabilities of said physical bioprocess machine helper (14) via said software-based bioprocess machine helper (14a).

11. The method according to claim 10, wherein said software-based bioprocess machine helper (14a) uses artificial intelligence algorithms, like machine learning models, neural networks, and others, and/or non-self-learning tools, like calculation models, for its predictions, computations, and data analyses.

12. The method according to claim 11, wherein the artificial intelligence algorithms are trained with either external data or the collected data about said at least one physico-chemical or biological properties from said biotechnological fluid in said physical biotechnological fluid treater (15).

13. The method according to claim 10, wherein the software-based bioprocess machine helper (14a) uses Smart Data for its predictions, computations, and data analyses.

14. The method according to claim 13, wherein the adaptive device creator (42) uses a contextualization mechanism on instances of said Smart data.

15. The method according to claim 10, wherein said software-based bioprocess machine helper (14a) uses learning models, neural networks, and/or calculation models, for predictions, computations, and data analyses.
